(19) United States
(12) Patent Application Publication
(10) Pub. No.: US 2020/0311652 A1
(43) Pub. Date: Oct. 1, 2020

(54) STAFFING AND PATIENT ACUITY TOOL

(71) Applicant: MaineHealth, Scarborough, ME (US)

(72) Inventor: Heidi E. Morin, S. Portland, ME (US)

(73) Assignee: MaineHealth, Scarborough, ME (US)

(21) Appl. No.: 16/812,500

(22) Filed: Mar. 9, 2020

Related U.S. Application Data
(60) Provisional application No. 62/816,092, filed on Mar. 9, 2019.

(51) Int. Cl. G06Q 10/06; G16H 40/20; G06N 20/00; G06Q 50/22

(52) U.S. Cl. CPC G06Q 10/063118; G06Q 50/22; G06N 20/00; G16H 40/20

(57) ABSTRACT

Embodiments of the innovation relate to a method for assigning staffing levels in a healthcare staffing device. The method includes displaying, by the healthcare staffing device, a set of patient acuity types associated with a department of a healthcare organization; receiving, by the healthcare staffing device, patient census information and patient acuity information for each patient acuity type of the set of patient acuity types associated with the department of the healthcare organization; applying, by the healthcare staffing device, staffing guidelines to the patient census information and patient acuity information for each patient acuity type; and outputting, by the healthcare staffing device, a staffing level indicator for the department of the healthcare organization based upon application of staffing guidelines to the patient census information and patient acuity information for each patient acuity type.

### RELATED APPLICATIONS

This patent application claims the benefit of U.S. Provisional Application No. 62/816,092 filed on Mar. 9, 2019 and entitled “Staffing and Patient Acuity Tool,” the contents and teachings of which are hereby incorporated by reference in their entirety.

### BACKGROUND

One of the many challenges facing healthcare organizations relates to nurse staffing. Many healthcare organizations utilize financial tracking tools to predict nurse staffing levels. For example, conventional financial tracking tools provide staffing estimates for one or more departments within the organization based upon historical data which relate worked nursing hours to a patient census carried by the department during those hours.

### SUMMARY

Conventional staff prediction tools suffer from a variety of deficiencies. As provided above, healthcare organizations utilize financial tracking tools using historic data to predict nurse staffing levels. However, healthcare organizations can experience variations in patient census and patient acuity levels over a relatively short time duration. Accordingly, in certain situations, the patient care needs can exceed the capacity of the nursing staff Inadequate staffing levels can lead to dissatisfaction among staff members.

Studies have shown that adequate levels of nurse staffing can increase patient safety. However, leaders have had difficulty articulating the need for adequate nurse staffing levels to meet the acuity needs of various patient populations in an objective and statistical manner. For example, the Guidelines for Professional Nurse Staffing for Perinatal Units by the Association of Women's Health, Obstetrics, and Neonatal Nursing (AWHONN, 2010) provide staffing recommendations based on patient acuity. However, the published staffing guidelines are complex and typically not well understood by healthcare administrators or executives.

By contrast to conventional staff estimation tools, embodiments of the present innovation relate to a staffing and patient acuity tool. In one arrangement, a healthcare staffing device is configured to receive patient census information via a graphical user interface (GUI) for a variety of patient acuity (e.g., severity of illness) types handled by a department within a healthcare organization. Based upon application of staffing guidelines to the patient census information, the healthcare staffing device can provide real-time recommendations for staffing needs of the department over a given time period.

The healthcare staffing device provides an easy-to-use frontline tool that gives the user the ability to compare the department's staffing levels with known, published staffing guidelines. As such, the healthcare staffing device mitigates the need for healthcare administrators or executives to be involved with staffing decisions or to understand complex guidelines. Further, the healthcare staffing device can track a department's ability to adjust their staff resources to match the patient volume and acuity levels in real time. Also, information provided by the healthcare staffing device can be used to improve the efficiency of resources, to advocate for additional resources, and to accurately and objectively capture the activity of the department.

In one arrangement, embodiments of the innovation relate to a method for assigning staffing levels in a healthcare staffing device. The method includes displaying, by the healthcare staffing device, a set of patient acuity types associated with a department of a healthcare organization; receiving, by the healthcare staffing device, patient census information and patient acuity information for each patient acuity type of the set of patient acuity types associated with the department of the healthcare organization; applying, by the healthcare staffing device, staffing guidelines to the patient census information and patient acuity information for each patient acuity type; and outputting, by the healthcare staffing device, a staffing level indicator for the department of the healthcare organization based upon application of staffing guidelines to the patient census information and patient acuity information for each patient acuity type.

In one arrangement, embodiments of the innovation relate to a healthcare staffing device, comprising a controller having a memory and a processor, the controller being configured to display a set of patient acuity types associated with a department of a healthcare organization; receive patient census information for each patient acuity type of the set of patient acuity types associated with the department of the healthcare organization; apply healthcare staffing device, staffing guidelines to the patient census information for each patient acuity type; and output a staffing level indicator for the department of the healthcare organization based upon application of staffing guidelines to the patient census information for each patient acuity type.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 illustrates a schematic representation of a healthcare staffing device, according to one arrangement.

FIG. 2 is a flowchart of a process performed by the healthcare staffing device of FIG. 1, according to one arrangement.

FIG. 3 illustrates a graphical user interface provided by the healthcare staffing device of FIG. 1, according to one arrangement.

FIG. 4 illustrates a report provided by the healthcare staffing device of FIG. 1, according to one arrangement.

FIG. 5A illustrates a productivity trend report, according to one arrangement.

FIG. 5B illustrates a productivity trend report, according to one arrangement.

FIG. 6A illustrates a worked hours per patient day trend report, according to one arrangement.

FIG. 6B illustrates a worked hours per patient day trend report, according to one arrangement.

FIG. 7 illustrates a feedback report, according to one arrangement.

FIG. 8 illustrates a schematic representation of a healthcare staffing device, according to one arrangement.

FIG. 9 illustrates a schematic representation of a healthcare staffing device configured to provide a graphical user interface for each unit of a department of a healthcare organization, according to one arrangement.

FIG. 10A illustrates a schematic representation of a healthcare staffing system, according to one arrangement.

FIG. 10B illustrates a schematic representation of a healthcare staffing system, according to one arrangement.

FIG. 11 illustrates an acuity tracking report provided by the healthcare staffing device of FIG. 1, according to one arrangement.

FIG. 12A illustrates a trended data report provided by the healthcare staffing device of FIG. 1, according to one arrangement.

FIG. 12B illustrates a trended data report provided by the healthcare staffing device of FIG. 1, according to one arrangement.

### DETAILED DESCRIPTION

Embodiments of the present innovation relate to a staffing and patient acuity tool. In one arrangement, a healthcare staffing device is configured to receive patient census information via a graphical user interface (GUI) for a variety of patient acuity types handled by a department within a healthcare organization. Based upon application of staffing guidelines to the patient census information, the healthcare staffing device can provide real-time recommendations for staffing needs of the department over a given time period.

The healthcare staffing device provides an easy-to-use frontline tool that gives the user the ability to compare the department's staffing levels with known staffing guidelines. As such, the healthcare staffing device mitigates the need for healthcare administrators or executives to be involved with staffing decisions or to understand complex guidelines. Further, the healthcare staffing device can track a department's ability to adjust their staff resources to match the patient volume and acuity levels in real time. Also, information provided by the healthcare staffing device can be used to improve the efficiency of resources, to advocate for additional resources, and to accurately and objectively capture the activity of the department.

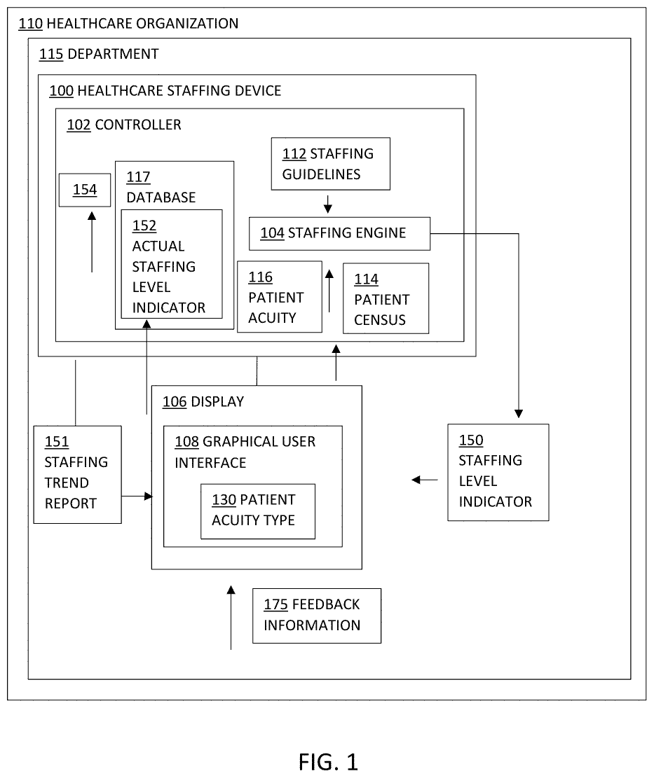

FIG. 1 illustrates a healthcare staffing device 100 which includes a controller 102, such as a processor and a memory, configured to execute a staffing engine 104. The staffing engine 104 configures the healthcare staffing device 100 to identify and recommend staffing levels for a department of a healthcare facility based upon patient census 114 and patient acuity 116 information received by the healthcare staffing device 100.

In one arrangement, the staffing engine 104 is configured to apply staffing guidelines 112 to the patient census 114 and patient acuity 116 information in order to identify staffing levels 150, such as nursing staffing levels, for a department 115 of a healthcare organization 110. The staffing guidelines 112 can provide suggestions regarding optimal staffing levels based upon the patient census and acuity for a department 115. For example, the staffing guidelines 112 (e.g., nursing staffing guidelines) can indicate that, in the presence of a given number of patients with a particular severity of illness, the department 115 should utilize a certain number of healthcare workers (e.g., nurses). With application of the staffing guidelines 112 to the patient census 114 and patient acuity 116 information, the staffing engine 104 can predict a department's staffing needs over a given timeframe.

In one arrangement, the staffing engine 104 is configured to utilize a set of staffing guidelines 112 in order to identify staffing levels for a single department 115 of a healthcare organization 110. A healthcare organization 110 can include multiple departments (e.g., surgery, trauma, obstetrics, etc.) where each department 115 has its own unique set of staffing guidelines which can identify optimum staffing levels based upon the patient census and acuity for that department. For example, a maternity/obstetrics department within the healthcare organization 110 can utilize, as the staffing guidelines 112, the Guidelines for Professional Nurse Staffing for Perinatal Units provided by the Association of Women's Health, Obstetrics, and Neonatal Nursing (AWHONN, 2010). However, a surgical department within the healthcare organization 110 can utilize a separate and distinct set of surgical staffing guidelines 112. Accordingly, in one arrangement, in order to apply the appropriate staffing guidelines 112 to the patient census 114 and patient acuity 116 information for a given department 115, each department 115 of the healthcare organization 110 can include a dedicated healthcare staffing device 100 configured with the staffing guidelines 112 for that particular department.

With continued reference to FIG. 1, the healthcare staffing device 100 is disposed in electrical communication with a display 106 and is configured to provide a graphical user interface (GUI) 108 to the display 106. The GUI 108 is configured to allow one or more users, such as healthcare workers, to interact with the healthcare staffing device 100 by inputting patient census information 114 for various patient acuity types 130 associated with the department 115. As will be described in detail below, the GUI 108 provides the user with an ease-of-use, such that the user can spend a relatively minimal amount of time to input information 114 into the healthcare staffing device 100 in order to identify the staffing levels for the department.

As a result of receiving the inputted patient census information 114 and associated patient acuityl16 information, the healthcare staffing device 100 can predict staffing levels for the department 115 in substantially real time. For example, the healthcare staffing device 100 can display the staffing levels as part of the GUI 108.

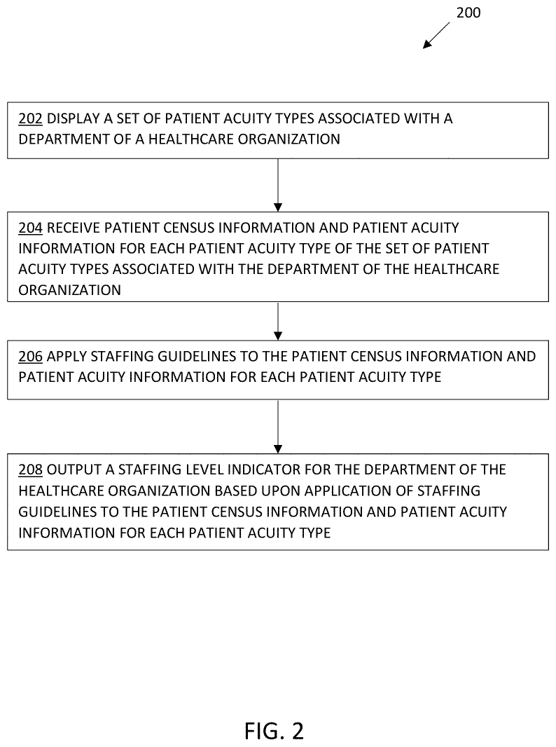

FIG. 2 is a flowchart 200 of a process performed by the healthcare staffing device 100 when providing the staffing levels.

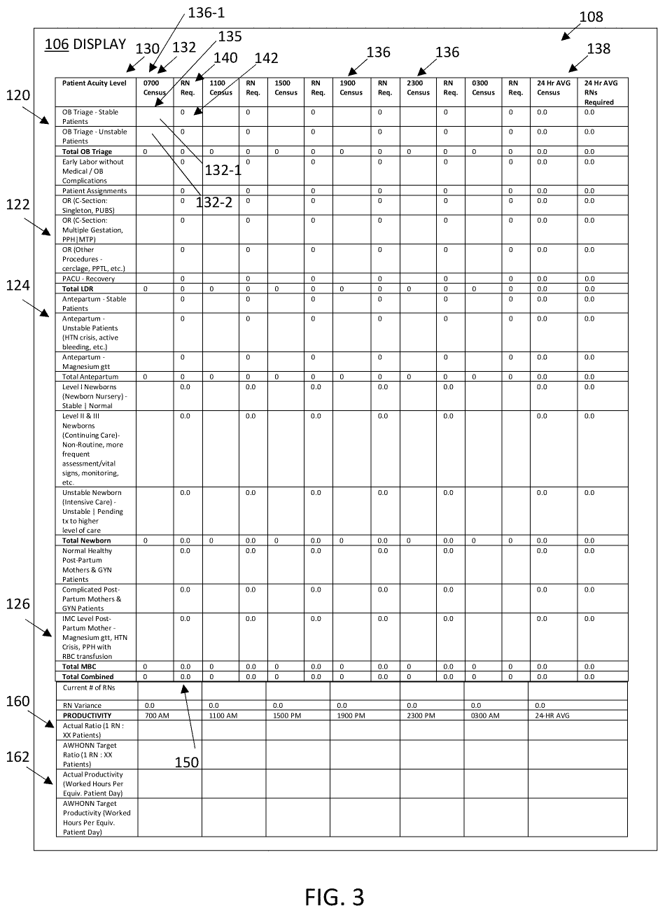

In element 202, the healthcare staffing device 100 displays a set of patient acuity types 130 associated with a department 115 of a healthcare organization 110. For example, FIG. 3 illustrates a sample GUI 108 provided by the healthcare staffing device 100 for a labor and delivery department. In one arrangement, the GUI 108 includes a listing of each of the units which constitutes the labor and delivery department. For example, the GUI 108 indicates that the labor and delivery department for a given healthcare organization 110 includes an OB triage unit 120, a labor and delivery unit 122, an antepartum unit 124, and a post-partum unit 126. The GUI 108 further provides a listing of the patient acuity types 130 associated with each unit. For example, for the OB triage unit 120, the GUI lists “OB Triage-Stable Patients” and “OB Triage-Unstable Patients” as the patient acuity types. With such a listing, the user can identify the patient acuity types 130 associated with each unit 120 through 126.

Returning to FIG. 2, in element 204, the healthcare staffing device 100 receives patient census 114 and patient acuityl16 information for each patient acuity type of the set of patient acuity types 130 associated with the department 115 of the healthcare organization 110. In one arrangement, with reference to FIG. 3, the GUI 108 provides patient census information cells 135 which allows the user to input patient census values 132 for each of the patient acuity types 130 provided. For example, a user can input a first patient census value 132-1 for the “OB Triage-Stable Patients”, a second patient census value 132-2 for “OB Triage-Unstable Patients”, and additional patient census values for each corresponding patient acuity type of the set of patient acuity types 130 displayed as part of the GUI 108. Following the input of patient census values 132 into cells 135, the healthcare staffing device 100 identifies the patient census values 132 as patient census information 114 and the corresponding patient acuity types 130 as patient acuity information 116, shown in FIG. 1.

In one arrangement, as indicated in FIG. 3, the GUI 108 is configured to provide patient census information cells 135 across multiple time divisions 136 of a given time period 138 associated with the department 115 of the healthcare organization 110. For example, the time period 138 for the department 115 can be defined as a twenty-four hour period (i.e., one full day). As such, the GUI 108 can display six, four-hour time divisions 136 where each of the six time divisions 136 can include patient census information cells 135 corresponding to each of the patient acuity types 130. As provided above, the user can input patient census information 132 within each of the patient census information cells 135 for each of the patient acuity types 130 provided and for each of the time divisions 136.

Returning to FIG. 2, in element 206, the healthcare staffing device 100 applies staffing guidelines 112 to the patient census information 114 and patient acuity information 116 for each patient acuity type 130. As indicated above, the staffing guidelines 112 can be unique to each department 115 in the healthcare organization 110. For example, for a maternity/obstetrics department, the staffing device 100 is configured to apply, as the staffing guidelines 112, AWHONN staffing guidelines to the patient census information 114 received from the patient census information cells 135 for each patient acuity type 130. The staffing guidelines 112 typically provide suggestions for department staffing based upon a number of factors, including patient acuity. For example, for “OB Triage—Stable Patients,” the AWHONN staffing guidelines recommend one nurse for two to three patients while for “OB Triage—Unstable Patients,” the AWHONN staffing guidelines recommend one nurse for every one patient.

In one arrangement, when applying the staffing guidelines 112 to the information 114, 116, the healthcare staffing device 100 can compare the patient census information 114 and patient acuity information 116 with the staffing guidelines 112 to identify the staffing needs of each unit of the department 115. For example, with reference to the first time division 136-1, assume the case where the patient acuity information 116 identifies “OB Triage—Unstable Patients” as the patient acuity type 130 and the patient census information 114 identifies three patients for that patient acuity type 130. With the AWHONN staffing guidelines identifying one nurse for one patient, the healthcare staffing device 100 can provide a recommendation of three nurses to care for the three patients classified as “OB Triage—Unstable Patients” during the first time division 136-1.

In one arrangement, by applying the staffing guidelines 112 to the patient census information 114 and patient acuity information 116, the healthcare staffing device 100 can identify recommended staffing level indicator values 142 for the department 115 on a per unit basis (i.e., for each of the OB triage unit 120, labor and delivery unit 122, etc.) and on a per time division 136 basis. For example, with reference to FIG. 3, the healthcare staffing device 100 can display, as part of the GUI 108, staffing information cells 140 across multiple time divisions 136 of a given time period 138 for each unit associated with the department 115 of the healthcare organization 110. Based on application of the staffing guidelines 112, the healthcare staffing device 100 can display the recommended staffing level indicator values 142 in the corresponding information cells 140.

Returning to FIG. 2, in element 208, the healthcare staffing device 100 outputs a staffing level indicator 150 for the department 115 of the healthcare organization 110 based upon application of staffing guidelines 112 to the patient census information 114 and patient acuity information 116 for each patient acuity type 130. For example, with reference to FIG. 3, the GUI 108 can display a staffing level indicator 150 for the department 115 each patient acuity type 130 and for each time division 136 of the time period 138.

In one arrangement, the healthcare staffing device 100 is configured to provide productivity measurements related to the patient census information 132 for each patient acuity type 130. For example, healthcare staffing device 100 can provide nurse-patient ratios 160 and worked hours per equivalent patient day (WHPPD) 162 values as part of the GUI 108.

With this process, the healthcare staffing device 100 is configured to provide a real-time recommendation of staffing needs for a department 115 for each time division 136, or shift, of the time period 138. User interaction with the GUI 108 is relatively non-labor intensive and, as such, the healthcare organization 110 can provide staffing recommendations as it receives the patient census values 132 for each patient acuity type 130. Further, the healthcare staffing device 100 can provide dynamic recommendations for staffing needs as it receives updated patient census values 132 for each patient acuity type 130 during the time period 138.

In one arrangement, the healthcare staffing device 100 is further configured to collect and store the staffing level indicator 150, along with additional information, over a series of time periods 138 for the department 115. For example, for each time division 136 of the time period 138, the healthcare staffing device 100 can store the patient census information 114, patient acuity information 116, and the calculated staffing level indicator 150 as part of a database 117. In one arrangement, for each calculated staffing level indicator 150 stored in the database 117, the healthcare staffing device 100 is configured to store an associated actual staffing level indicator 152. For example, when the GUI 108 displays the staffing level indicator 150 for each patient acuity type 130 and each time division 136, the GUI 108 allows the operator to enter the actual number of staff members (e.g., nurses) assigned to the patients, as well as hours worked, in light of the recommendation. The GUI 108, in turn, provides each entry to the healthcare staffing device 100 as the actual staffing level indicator 152.

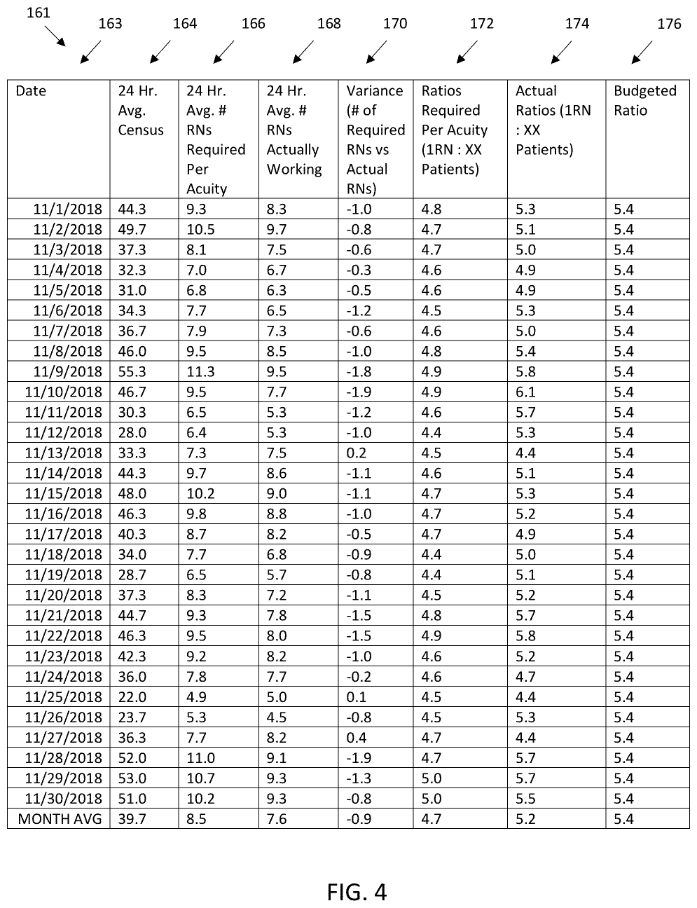

The healthcare staffing device 100 can access the database 117 to generate a variety of staffing reports 161. For example, with reference to FIG. 4, based upon such a collection of information 114, 116 for the department 115 over successive time periods (e.g., days) 138 for one month, the healthcare staffing device 100 can generate and output a staffing report 161 which offers insight into the staffing and operation of the department 115 during the course of the month. As illustrated in FIG. 4, the staffing report 161 includes information collected or derived by the healthcare staffing device 100 over the course of one month, as indicated by a date identifier 163. For each date identified by the date identifier 163, the staffing report 161 can include information relating to the average patient census over a time period 164, an average number of nurses required to work during the time period 166, an average number of nurses actually working during the time period 168, the variance between required and actual working nurses 170, the nurse-patient ratio required per acuity 172, the actual nurse-patient ratio 174, and the budgeted ratio 176.

In another example, based upon the collection of information for the department 115 over successive time periods (e.g., days, months. etc.) 138 in the database 117, the healthcare staffing device 100 is configured to detect staffing level trends 154 for the department 115 and to generate a staffing trend report 151.

For example, as indicated in FIG. 1, the healthcare staffing device 100 can access the database 117 to retrieve the staffing level indicator 150 for each patient acuity type 130 and each time division 136, as well as the actual staffing level indicator 152 for each patient acuity type 130 and each time division 136. By identifying changes in the staffing level indicator 150 for each subsequent time division 136, as well as by identifying changes in the actual staffing level indicator 152 for each time division 136, the healthcare staffing device 100 can detect the trends in the staffing for the department 115 over the time period 138. Based upon the detected trends, the healthcare staffing device 100 can provide a staffing trend report 151 to a user via the GUI 108 where the report 151 identifies deviations between the actual staffing levels provided by the actual staffing level indicators 152 and the recommended staffing level indicator values 142 provided by the staffing level indicators 150.

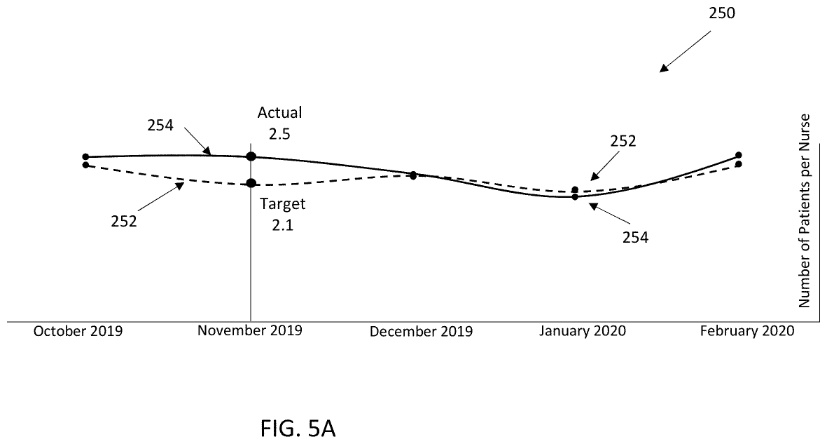

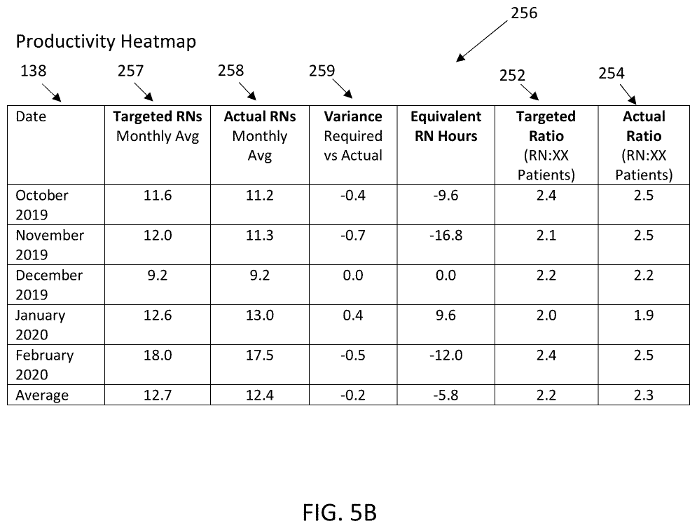

The staffing trend reports 151 can be configured to provide a variety of types of information. In one arrangement, as indicated in FIG. 5A, the healthcare staffing device 100 is configured to generate, as the staffing trend report 151, a productivity trend report 250 which identifies the productivity trend for the department 115 based on a ratio of number of patients supported per nurse. The productivity trend report 250 can identify a target productivity ratio trend 252 for a time period 138, such as between October 2019 and February 2020 as shown, and an actual productivity ratio trend 254 for the same time period 138. As shown, the productivity trend report 250 indicates that prior to December 2019, the actual productivity ratio 254 is ahead of the target productivity ratio 252 and after December 2019, the actual productivity ratio 254 is behind the target productivity ratio 252. Based upon this trend, the department 115 can adjust staffing such that the actual productivity for a given time matches the target productivity.

In one arrangement, as indicated in FIG. 5B, the healthcare staffing device 100 can generate, as the staffing trend report 151, a productivity heatmap report 256 which identifies nursing productivity for a given time period 138. For example, the heatmap report 256 can provide the actual productivity ratio 254 and the target productivity ratio 252 for each time division 136 within the time frame 138. The heatmap report 256 can also provide a monthly average of the targeted number of nurses working 257 per time division 136, a monthly average of the actual number of nurses working 258 per time division 136, and the variance of the monthly average of the targeted to actual number of nurses working 259.

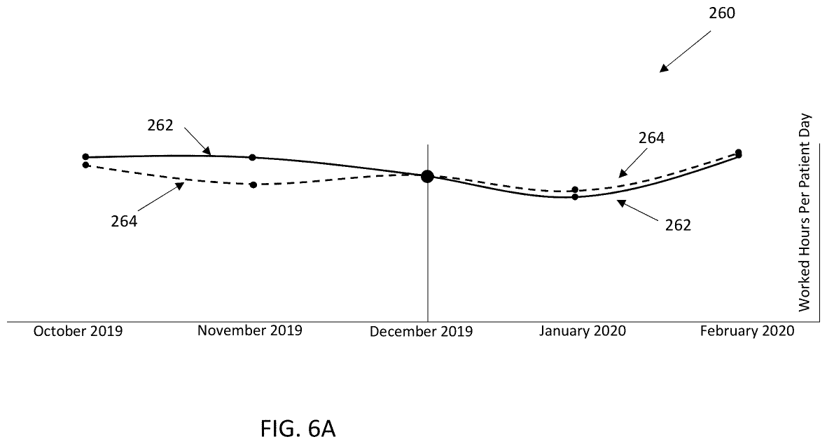

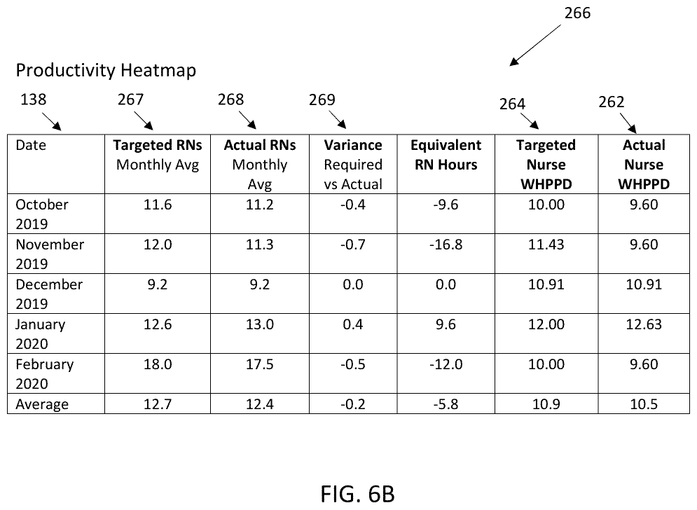

In one arrangement, as indicated in the FIG. 6A, the healthcare staffing device 100 can access the database 117 to generate, as the staffing trend report 151, a worked hours per patient day (WHPPD) trend report 260. The WHPPD report 260 can identify a target worked hours per day trend 262 for a time period 138, such as between October 2019 and February 2020 as shown, and an actual worked hours per day trend 264 for the same time period 138. As shown, the WHPPD trend report 260 indicates that prior to December 2019, the actual worked hours per day 262 is behind the target worked hours per day 264 and after December 2019, the actual worked hours per day 262 is ahead of the target worked hours per day 262. Based upon this trend, the department 115 can adjust staffing such that the actual worked hours per day for a given time matches the target worked hours per day.

In one arrangement, as indicated in FIG. 6B, the healthcare staffing device 100 can generate, as the staffing trend report 151, a productivity heatmap report 266 which identifies the worked hours per patient day (WHPPD) for a given time period 138. For example, the heatmap report 266 can provide the target worked hours per day 264 and actual worked hours per day 262 for each time division 136 within the time frame 138. The heatmap report 266 can also provide a monthly average of the targeted number of nurses working 267 per time division 136, a monthly average of the actual number of nurses working 268 per time division 136, and the variance of the monthly average of the targeted to actual number of nurses working 269.

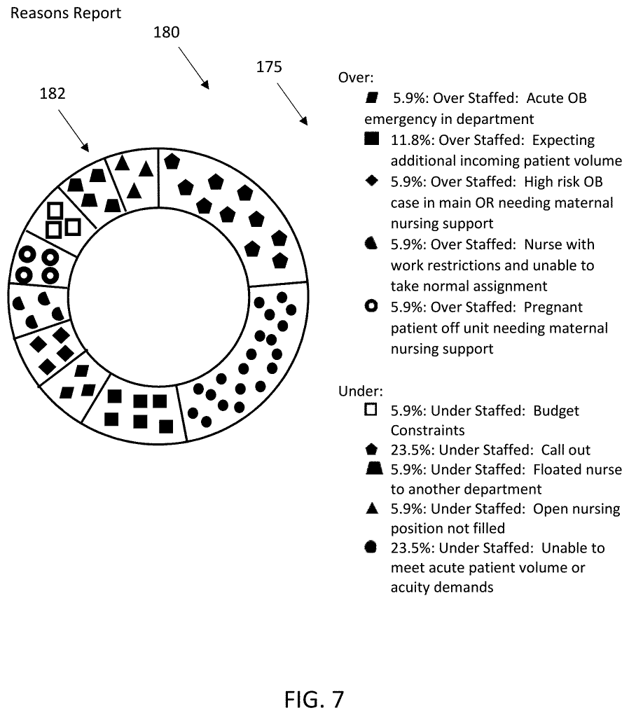

In one arrangement, after generating and providing the staffing trend report 151 to the GUI 108, the healthcare staffing device 100 can be configured to receive feedback information regarding aspects of the report 151 from the end user via the GUI 108. For example, with reference to FIG. 1, assume the case where the staffing trend report 151 identifies, for each acuity type 130 and for each time division 136 of a time period 138, a variance between the targeted number of nurses working and the actual number of nurses working. In such a case, the end user can enter feedback information 175 into the GUI 108 which describes the reasoning for one or more of the variances (e.g., over/under) identified between the targeted and actual number of nurses working. As the healthcare staffing device 100 receives the feedback information 175 from the GUI 108, the healthcare staffing device 100 can associate the feedback information 175 with a given patient acuity type 130 and time division 136 and can generate a feedback report 180, such as illustrated in FIG. 7, identifying both variance information 182 and feedback information 175.

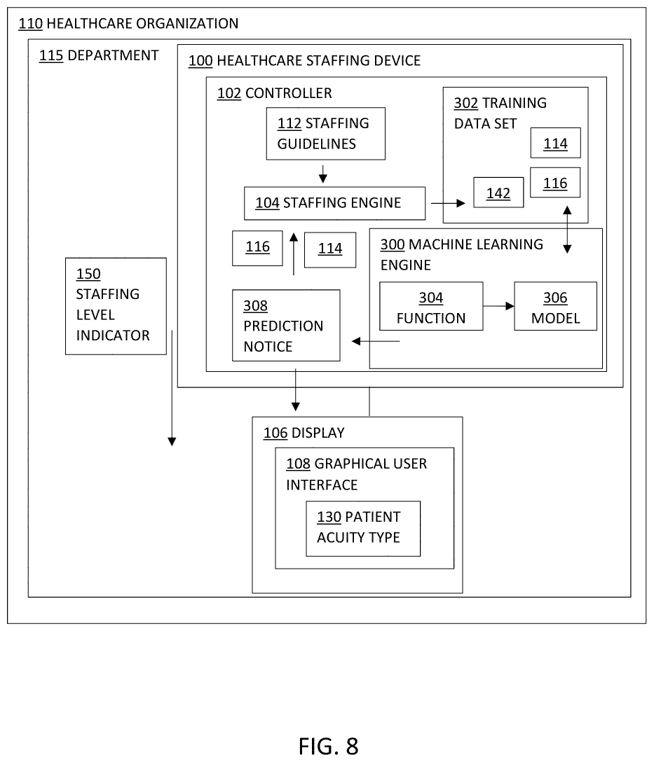

In one arrangement, the healthcare staffing device 100 can be configured to predict staffing levels for the department 115 based upon the patient census information 114 for each patient acuity type 130 over the successive time periods 138. For example, with reference to FIG. 8, the healthcare staffing device 100 can include a machine learning engine 300 which is configured to build a training data set 302 for a given department 115 over a given time period 138 (e.g., month, year, etc.). During operation, for each successive time division 136 of the time period 138, the machine learning engine 300 can generate a training data set 302 which includes patient census information 114, corresponding patient acuity type information 116, and recommended staffing level values 142 for each acuity type 130, based upon the staffing level indicators 150.

With the healthcare staffing device 100 having developed the training data set 302 for a particular department 115, the machine learning engine 300 can train a statistical function 304 with the training data set 302 to generate a staffing prediction model 306. For example, the machine learning engine 300 can fit a statistical function 304, such as a Random Forest, neural network, or deep learning function to the training data set 302 to develop the prediction model 306.

Once developed, the machine learning engine 300 can utilize the model 306 to predict future behavior of the department 115 during subsequent time divisions 136 in response to receiving additional or updated patient census 114 or patient acuity 116 information. In one arrangement, based upon the application of updated patient census 114 or patient acuity 116 information to the model 306, the machine learning engine 300 can identify a predicted staffing level 308 from the staffing prediction mode 306 where the predicted staffing level 308 indicates a staffing level for the department 115 of the healthcare organization 110 for at least one of a subsequent time division 136 and a subsequent time period 138.

For example, based upon use of the model 306, the machine learning engine 300 can identify time divisions 136 which are typically understaffed and can transmit the predicted staffing level 308 as a prediction notice to the GUI 108 which provides a recommendation regarding staffing levels for subsequent time divisions 138. In another example, the machine learning engine 300 can receive additional or updated patient census 114 or patient acuity 116 information and can apply the information 114, 116 to the model 306 to predict the patient acuity types 130 to be experienced by the department 115 for a given time of day, time of week, or time of year. Since the model 306 is developed in light of known staffing guidelines 112, such as

AWHONN staffing guidelines, predictions resulting from application of the model can also be considered as compliant with such staffing guidelines.

As provided above, each department 115 can include its own healthcare staffing device 100. In such a case, the single healthcare staffing device 100 can display a GUI 108 which identifies patient census 114 and patient acuity 116 information for all units within the department 115. In one arrangement, in the case where the healthcare organization is relatively large, healthcare staffing device 100 can divide the GUI 108 into different components and can provide each component to separate displays 106 within the department 115.

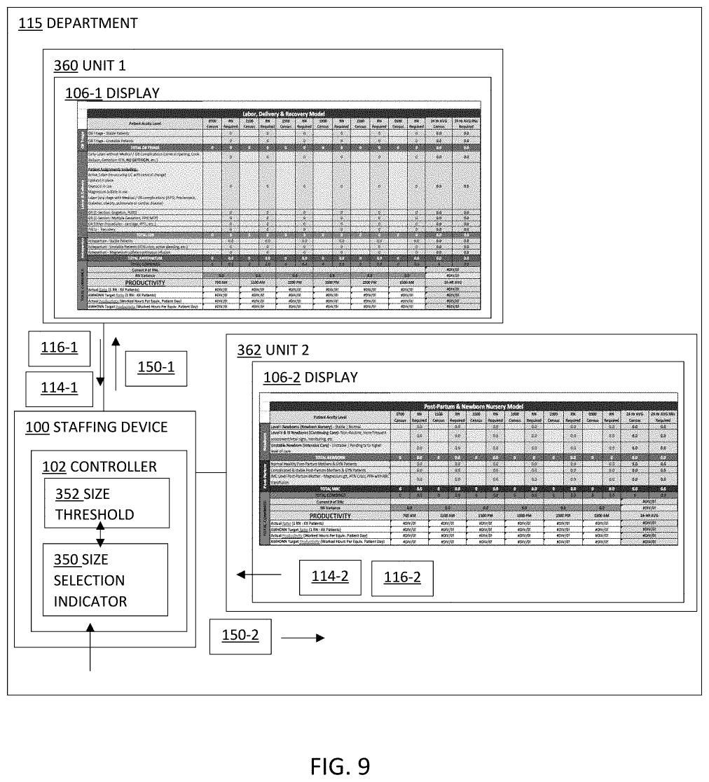

For example, with reference to FIG. 9, the healthcare staffing device 100 is configured to receive a size selection indicator 350 which identifies a size of the healthcare organization 110. In the case where the healthcare staffing device 100 detects the size selection indicator 350 as exceeding a size threshold 352 (i.e., which indicates the healthcare organization 110 as being relatively large), the healthcare staffing device 100 is configured to identify the set of units associated with the department 115 of the healthcare organization 110. For example, assume the case where the department 115 is a maternity/obstetrics department. In such a case, the healthcare staffing device 100 can identify the maternity/obstetrics department as having a labor, delivery, and recovery unit 360 and a post-partum and newborn nursery unit 362.

Following this identification, the healthcare staffing device 100 can divide the GUI 108 into components based upon the identified units and can direct the GUI components to the corresponding displays 106 in the units 360, 362. For example, the healthcare staffing device 100 can provide a first GUI component 108-1 identifying a labor, delivery, and recovery model to a first display 106-1 in the labor, delivery, and recovery unit 360 and can provide a second GUI component 108-2 identifying a post-partum and newborn nursery model to a second display 106-1 in the post-partum and newborn nursery unit 36. As such, the healthcare staffing device 100 displays the set of patient acuity types 130 associated with each respective unit 360, 362 of the healthcare organization 110. During operation, an operator, such as a nurse manager, for each respective unit 360, 362 can provide patient census information 114-1, 114-2 and patient acuity information 116-1, 116-2, respectively, for each patient acuity type 130 associated with that unit 360, 362 to the healthcare staffing device 100. The healthcare staffing device 100 can then apply staffing guidelines 112 to the patient census information 114-1, 114-2 and patient acuity information 116-1, 116-2 for each unit 360, 362 and output a corresponding staffing level indicator 150-1, 150-2 for each patient acuity type 130 associated with each unit 360, 362 of the healthcare organization 110.

As indicated in FIG. 9, one healthcare staffing device 100 can be utilized for multiple units of a department 115. However, it should be understood that each unit within a department 115 can include its own dedicated healthcare staffing device 100.

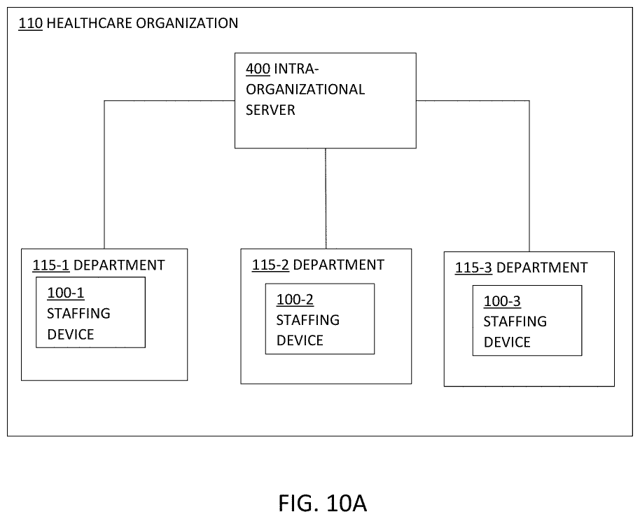

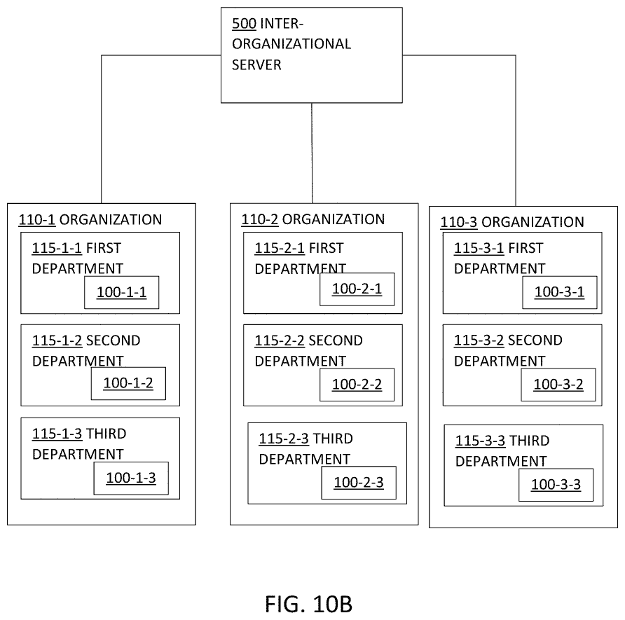

As provided above, a healthcare staffing device 100 is utilized to collect patient census 114 and patient acuity 116 information for a given department 115 a healthcare organization 110. However, a more global collection process can be utilized. For example, with reference to FIG. 10A, an intra-organization server 400 can be configured to collect patient census 114 and patient acuity 116 information from multiple departments, such as departments 115-1 through 115-3, in the healthcare organization 110 where each department 115 includes a dedicated healthcare staffing device 100-1 through 100-3. In another example, with reference to FIG. 10B, an inter-organizational server 500 can be can be configured to collect patient census 114 and patient acuity 116 information from multiple healthcare organizations, such as healthcare organizations 110. In either example, the intra-organization server 400 or the inter-organizational server 500 can apply staffing guidelines 112 to the collected information 114 116 and can provide staffing recommendations, trend analysis, and predictions on a global level.

As provided above, the healthcare staffing device 100 can generate and output a staffing report 161 which offers insight into the staffing and operation of the department 115 during the course of the month. In one arrangement, the healthcare staffing device 100 can generate a variety of reports. For example, these reports can provide a user with real-time information to flex nursing resources appropriately and safely. Additionally, the reports can provide nursing productivity information (i.e., nursing ratios which identifies the number of patients per nurse) over any time period to allow an end user to make longer-term operating decisions with respect to the department 115, such as to increase or decrease number of budgeted nurses. The reports can also be used to provide information used to improve the efficiency of resources within a department, to advocate for additional resources, and to accurately and objectively capture the activity of the department 115.

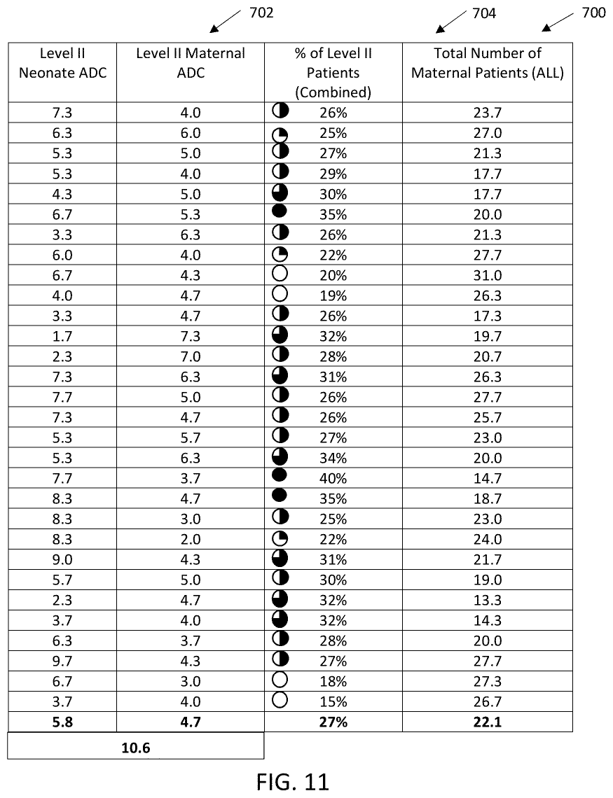

In one example, as shown in FIG. 11, the healthcare staffing device 100 can provide a acuity tracking report 700 based upon the tracking of a given number (e.g., Average Daily Census) of a department's higher risk maternal and neonatal patients. The acuity tracking report 700 indicates the percentage of the department patient volume that is at a relatively high risk. Accordingly, the report 700 can provide the staff with a better understanding of the proportion of patients receiving care in a department 115.

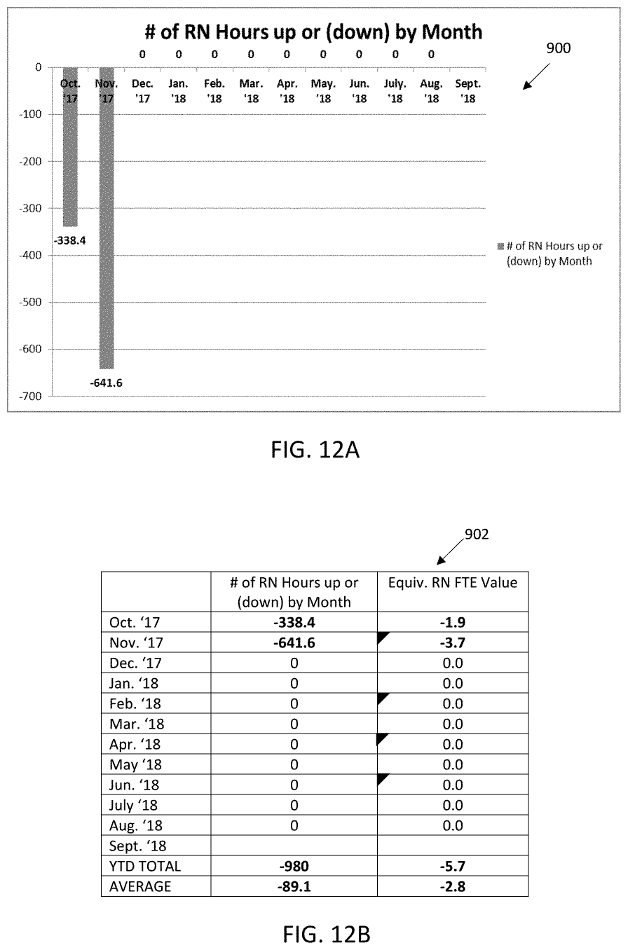

In another example, as shown in FIG. 12A, the healthcare staffing device 100 can generate a report 800 that provides a graphical representation of data collected for a department 115, such as ratios required per acuity versus the actual acuities over the course of the month. Further, as shown in FIG. 12B, the healthcare staffing device 100 can generate trended data reports 900. These reports 800, 900 can provide information which identifies the impact of nursing productivity and can provide organization leaders with information to make resource/labor decisions, such as hiring of additional staff, attrition of staff, or re-allocate of staff, for example.

While various embodiments of the innovation have been particularly shown and described, it will be understood by those skilled in the art that various changes in form and details may be made therein without departing from the spirit and scope of the innovation as defined by the appended claims.

1. In a healthcare staffing device, a method for identifying staffing levels, comprising:
displaying, by the healthcare staffing device, a set of patient acuity types associated with a department of a healthcare organization;
receiving, by the healthcare staffing device, patient census information and patient acuity information for each patient acuity type of the set of patient acuity types associated with the department of the healthcare organization;
applying, by the healthcare staffing device, staffing guidelines to the patient census information and patient acuity information for each patient acuity type; and
outputting, by the healthcare staffing device, a staffing level indicator for the department of the healthcare organization based upon application of staffing guidelines to the patient census information and patient acuity information for each patient acuity type.

2. The method of claim 1, wherein:
receiving patient census information for each patient acuity type comprises receiving, by the healthcare staffing device, patient census information for each patient acuity type for at least one time division of a time period associated with the department of the healthcare organization; and
outputting the staffing level indicator for the department of the healthcare organization comprises outputting, by the healthcare staffing device, the staffing level indicator for each patient acuity type for the at least one time division of the time period associated with the department of the healthcare organization based upon application of staffing guidelines to the patient census information and patient acuity information for each patient acuity type.

3. The method of claim 1, wherein applying staffing guidelines to the patient census information for each patient acuity type comprises applying, by the healthcare staffing device, Guidelines for Professional Nurse Staffing for Perinatal Units provided by the Association of Women's Health, Obstetrics, and Neonatal Nursing to the patient census information for each patient acuity type.

4. The method of claim 1, wherein:
displaying the set of patient acuity types associated with a department of a healthcare organization comprises displaying, by the healthcare staffing device, the set of patient acuity types associated with the department of the healthcare organization via a graphical user interface (GUI);
receiving patient census information and patient acuity information comprises receiving, by the healthcare staffing device, patient census information and patient acuity information for each patient acuity type of the set of patient acuity types associated with the department of the healthcare organization via the GUI; and
outputting the staffing level indicator for the department of the healthcare organization comprises outputting, by the healthcare staffing device, the staffing level indicator for the department of the healthcare organization via the GUI.

5. The method of claim 1, further comprising identifying, by the healthcare staffing device, a staffing level trend for the department of the healthcare organization based upon at least one of the staffing level indicator and an actual staffing level indicator for each patient acuity type over the at least one time period.

6. The method of claim 5, further comprising receiving, by the healthcare staffing device, feedback information from an end user, the feedback information identifying reasoning for variance in the staffing level trend for the department of the healthcare organization.

7. The method of claim 1, further comprising:
generating, by the healthcare staffing device, a staffing prediction model based upon a training data set which includes patient census information, corresponding patient acuity type information, and recommended staffing level values for each acuity type;
applying, by the healthcare staffing device, at least one of updated patient census information and updated patient acuity information and to the staffing prediction model for a patient acuity type;
identifying, by the healthcare staffing device, a predicted staffing level from the staffing prediction model, the predicted staffing level indicating a staffing level for the department of the healthcare organization for at least one of a subsequent time division and a subsequent time period.

8. The method of claim 7, wherein generating the staffing prediction model comprises:
developing, by the healthcare staffing device, the training data set from the patient census information, the patient acuity information for each patient acuity type of the set of patient acuity types associated with the department of the healthcare organization, and recommended staffing level values for each acuity type based upon the staffing level indicator; and
training, by the healthcare staffing device, a statistical function with the training data set to generate the staffing prediction model.

9. The method of claim 1, further comprising:
identifying, by the healthcare staffing device, a set of units associated with the department of the healthcare organization; and wherein:
displaying the set of patient acuity types associated with the department of the healthcare organization comprises displaying on a display at each unit, by the healthcare staffing device, the set of patient acuity types associated with each respective unit of the healthcare organization;
receiving patient census information and patient acuity information for each patient acuity type of the set of patient acuity types associated with the department of the healthcare organization comprises receiving, by the healthcare staffing device, patient census information and patient acuity information for each patient acuity type of the set of patient acuity types associated with each unit of the healthcare organization;
applying staffing guidelines to the patient census information and patient acuity information for each patient acuity type comprises applying, by the healthcare staffing device, staffing guidelines to the patient census information and patient acuity information for each patient acuity type associated with each unit of the healthcare organization; and
outputting the staffing level indicator for the department of the healthcare organization based upon application of staffing guidelines to the patient census information and patient acuity information for each patient acuity type comprises outputting, by the healthcare staffing device, the staffing level indicator for each unit of the healthcare organization based upon application of staffing guidelines to the patient census information and patient acuity information for each patient acuity type associated with each unit of the healthcare organization.

10. The method of claim 9, wherein identifying the set of units associated with the department of the healthcare organization comprises:
receiving, by the healthcare staffing device, a size selection indicator which identifies a size of the healthcare organization; and
when the size selection indicator exceeds a size threshold, identifying, by the healthcare staffing device, the set of units associated with the department of the healthcare organization.

11. A healthcare staffing device, comprising:
a controller having a memory and a processor, the controller configured to:
display a set of patient acuity types associated with a department of a healthcare organization;
receive patient census information for each patient acuity type of the set of patient acuity types associated with the department of the healthcare organization;
apply healthcare staffing device, staffing guidelines to the patient census information for each patient acuity type; and
output a staffing level indicator for the department of the healthcare organization based upon application of staffing guidelines to the patient census information for each patient acuity type.

12. The healthcare staffing device of claim 11, wherein:
when receiving patient census information for each patient acuity type, the controller is configured to receive patient census information for each patient acuity type for at least one time division of a time period associated with the department of the healthcare organization; and
when outputting the staffing level indicator for the department of the healthcare organization, the controller is configured to output the staffing level indicator for each patient acuity type for the at least one time division of the time period associated with the department of the healthcare organization based upon application of staffing guidelines to the patient census information and patient acuity information for each patient acuity type.

13. The healthcare staffing device of claim 11, wherein when applying staffing guidelines to the patient census information for each patient acuity type the controller is configured to apply Guidelines for Professional Nurse Staffing for Perinatal Units provided by the Association of Women's Health, Obstetrics, and Neonatal Nursing to the patient census information for each patient acuity type.

14. The healthcare staffing device of claim 11, wherein:
when displaying the set of patient acuity types associated with a department of a healthcare organization the controller is configured to display the set of patient acuity types associated with the department of the healthcare organization via a graphical user interface (GUI);
when receiving patient census information and patient acuity information the controller is configured to receive patient census information and patient acuity information for each patient acuity type of the set of patient acuity types associated with the department of the healthcare organization via the GUI; and
when outputting the staffing level indicator for the department of the healthcare organization the controller is configured to output the staffing level indicator for the department of the healthcare organization via the GUI.

15. The healthcare staffing device of claim 11, wherein the controller is further configured to identify a staffing level trend for the department of the healthcare organization based upon at least one of the staffing level indicator and an actual staffing level indicator for each patient acuity type over the at least one time period.

16. The healthcare staffing device of claim 15, wherein the controller is further configured to receive feedback information from an end user, the feedback information identifying reasoning for variance in the staffing level trend for the department of the healthcare organization.

17. The healthcare staffing device of claim 11, wherein the controller is further configured to:
generate staffing prediction model based upon a training data set which includes patient census information, corresponding patient acuity type information, and recommended staffing level values for each acuity type;
apply at least one of updated patient census information and updated patient acuity information and to the staffing prediction model for a patient acuity type;
identify a predicted staffing level from the staffing prediction model, the predicted staffing level indicating a staffing level for the department of the healthcare organization for at least one of a subsequent time division and a subsequent time period.

18. The healthcare staffing device of claim 17, wherein when generating the staffing prediction model, the controller is further configured to:
develop the training data set from the patient census information, the patient acuity information for each patient acuity type of the set of patient acuity types associated with the department of the healthcare organization, and recommended staffing level values for each acuity type based upon the staffing level indicator; and
train a statistical function with the training data set to generate the staffing prediction model.

19. The healthcare staffing device of claim 11, wherein the controller is further configured to:
identify a set of units associated with the department of the healthcare organization; and wherein
when displaying the set of patient acuity types associated with the department of the healthcare organization, the controller is configured to display on a display at each unit, by the healthcare staffing device, the set of patient acuity types associated with each respective unit of the healthcare organization;
when receiving patient census information and patient acuity information for each patient acuity type of the set of patient acuity types associated with the department of the healthcare organization the controller is configured to receive patient census information and patient acuity information for each patient acuity type of the set of patient acuity types associated with each unit of the healthcare organization;
when applying staffing guidelines to the patient census information and patient acuity information for each patient acuity type the controller is configured to apply staffing guidelines to the patient census information and patient acuity information for each patient acuity type associated with each unit of the healthcare organization; and
when outputting the staffing level indicator for the department of the healthcare organization based upon application of staffing guidelines to the patient census information and patient acuity information for each patient acuity type, the controller is configured to output the staffing level indicator for each unit of the healthcare organization based upon application of staffing guidelines to the patient census information and patient acuity information for each patient acuity type associated with each unit of the healthcare organization.

20. The healthcare staffing device of claim 29, wherein when identifying the set of units associated with the department of the healthcare organization, the controller is configured to:
receive a size selection indicator which identifies a size of the healthcare organization; and
when the size selection indicator exceeds a size threshold, identify the set of units associated with the department of the healthcare organization.
